(19) United States
(12) Patent Application Publication
(10) Pub. No.: US 2011/0183312 A1
(43) Pub. Date: Jul. 28, 2011

(54) MICROFLUIDIC CHIP FOR ACCURATELY CONTROLLABLE CELL CULTURE

(75) Inventor: Yanyi Huang, Beijing (CN)

(73) Assignee: Peking University

(21) Appl. No.: 12/529,157

(22) PCT Filed: Aug. 29, 2008
(86) PCT No.: PCT/IB2008/002247

§ 371 Date: Aug. 28, 2009

(51) Int. Cl. C12Q 3/00; C12M 3/00; C12N 5/02; C12Q 1/02

(52) U.S. Cl. 435/3; 435/289.1; 435/286.5; 435/383; 435/29

(57) ABSTRACT

A device is disclosed for culturing cells in at least one cell culture well. The device includes one or more interconnecting layers having a pattern therein, the pattern including at least one microfluidic channel, at least one cell culture well having an opening at one end and a side wall, the at least one microfluidic channel in fluid communication with the side wall of the at least one cell culture well, and having a maximum channel width substantially less than a maximum width of the at least one cell culture well. The device includes at least one of a controllable valve and a controllable pump in fluid communication with the microfluidic channel, the valve and pump being configured to selectably restrict fluid transport through the microfluidic channel. In some embodiments, the device includes a removable top layer adapted to cover each of the at least one cell culture well.

### FIELD

The present technology relates to cell culturing. More particularly, the present technology relates to culturing cells in accessible microwells on a microfluidic chip.

### BACKGROUND

Cell culture is a fundamental step for biological and medical research. Present cell culture techniques are usually labor intensive and governed by a static cell culture growth medium, which is difficult to both monitor and control, as well as being counterintuitive to a biological process. Additionally, present cell culture techniques, in part due to the very static nature of the cell culture growth medium, require transferring a cell or group of cells under culture to multiple media, often with a pipette, at great risk of damage to the cell or group of cells under culture.

Microfluidic devices do allow for a dynamic medium for cell cultures, a medium that can be monitored and controlled, adjusting the nutrition and environment of the cell cultures dynamically by automatic or manual feedback means. However, microfluidic devices are presently limited to single cell cultures and very small groups of cells. Many microfluidic devices are not suitable for culturing embryonic cells, for example, and other large scale cells that cannot fit into the microchannels of the microfluidic devices. Further, some cells are too fragile, or the culture itself is too fragile, to expect a successful cell culture experiment given the shear and compressive forces imparted on a cell culture as it traverses the microchannels in the microfluidic devices. Fragility is a particular issue with stem cell cultures, for example.

### SUMMARY

The present technology provides an array of microwells patterned in a microfluidic chip to allow easy access to a cell or group of cells under culture. Patterned microwells in a microfluidic chip can bridge the technologies of microfluidic channels on a chip and a conventional biomedical experimental process that involves pipetting. Various embodiments can include a dynamically controlled fluidic environment for the array of microwells and a dynamically controlled atmospheric environment for the array of microwells.

In some embodiments, a device for culturing cells includes one or more interconnecting layers having a pattern therein, the pattern including at least one microfluidic channel. The device includes at least one cell culture well having an opening at one end and a side wall, the at least one microfluidic channel in fluid communication with the side wall of the at least one cell culture well, and having a maximum channel width substantially less than a maximum width of the at least one cell culture well.

In some embodiments, the one or more interconnecting layers include two or more interconnecting layers, wherein the at least one microfluidic channel is defined by the two or more interconnecting layers.

In some embodiments, the maximum width of the at least one cell culture well is at least ten times the maximum channel width. In some embodiments, the maximum width of the at least one cell culture well is at least sixty times the maximum channel width.

In some embodiments, the device includes an at least one controllable valve in fluid communication with the at least one microfluidic channel, the at least one controllable valve configured to selectably restrict fluid transport through the at least one microfluidic channel. In some embodiments, the controllable valve is a flexure valve.

In some embodiments, the device further includes at least one pump in fluid communication with the at least one microfluidic channel, the at least one pump configured to transport fluid through the at least one microfluidic channel. In some embodiments, the least one pump is a peristaltic pump.

In some embodiments, the one or more interconnecting layers includes polydimethylsiloxane (PDMS). In some embodiments, the device further includes a removable top layer adapted to cover each of the at least one cell culture well. In some embodiments, the at least one cell culture well contains at least one of a pluripotent cell. In some embodiments, the device further includes an externally accessible port in fluid communication with at least one of the at least one microfluidic channels.

In some embodiments, the device further includes at least one sensor in proximity to the at least one cell culture well and configured to observe a physical attribute of a set of test cells disposed within the at least one cell culture well. The device includes a controller in communication with the at least one sensor and at least one fluid flow regulator in communication with the controller. The controller controls the at least one fluid flow regulators, thereby regulating transportation of fluid through the at least one microfluidic channel responsive to the physical attribute observed by the at least one sensor.

In some embodiments, the device further includes a sensor selected from the group consisting of: an image sensor, a flow rate sensor, an ionic composition sensor, a temperature sensor, a pressure sensor, a light sensor, and a spectroscopic sensor.

In some embodiments, a process for culturing test cells includes transferring at least one set of test cells through an opening disposed at one end of at least one cell culture well and transporting a fluid through at least one microfluidic channel in fluid communication with a side wall of the at least one cell culture well. Each of the at least one microfluidic channels have a maximum channel width substantially less than a maximum width of the at least one cell culture well. The transported fluid promotes culturing of the at least one set of test cells disposed within the at least one cell culture well.

In some embodiments, the act of transporting fluid through the at least one microfluidic channel includes varying a pressure within the at least one microfluidic channel, the pressure variation driving fluid between the at least one microfluidic channel and a respective one of the at least one cell culture wells. In some embodiments, the varying a pressure within the at least one microfluidic channel includes pumping fluid through the at least one microfluidic channel. In some embodiments, the pumping fluid through the at least one microfluidic channel includes using at least one of a syringe and a peristaltic pump.

In some embodiments, the process further includes regulating by at least one valve the transportation of a fluid through the at least one microfluidic channel. In some embodiments, the process further includes measuring at least one parameter selected from the group consisting of: a microfluidic channel fluid velocity, a microfluidic channel fluid ionic composition, a cell culture well temperature, a cell culture well pressure, an optical transmissivity, an optical reflectivity, and a spectroscopic data of the cell culture well.

In some embodiments, the process further includes reversibly sealing a removable top layer adapted to cover the opening disposed at one end of each of the at least one cell culture wells. In some embodiments, the at least one set of test cells includes at least one of a pluripotent cell.

In some embodiments, the process further includes sensing a physical attribute of the at least one set of test cells disposed within the at least one cell culture well and regulating transportation of fluid through the at least one microfluidic channel responsive to the sensed physical attribute.

In some embodiments, a device for culturing cells includes means for storing at least one set of test cells, the at least one set of test sells being transferable through an opening disposed at one end, and means for transporting a fluid through at least one microfluidic channel in fluid communication with a side wall of the storing means. Each of the at least one microfluidic channels have a maximum channel width substantially less than a maximum width of the storing means, wherein the transported fluid promotes culturing of the at least one set of test cells disposed within the storing means.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other objects, features and advantages of the technology will be apparent from the following more particular description of embodiments of the technology, as illustrated in the accompanying drawings in which like reference characters refer to the same parts throughout the different views. The drawings are not necessarily to scale, emphasis instead being placed upon illustrating the principles of the technology.

FIG. 1 shows a cross-sectional side view of an illustrative embodiment of a device for culturing cells in accordance with an embodiment of the present technology.

FIG. 2 shows a cross-sectional side view of an illustrative embodiment of a device for culturing cells in accordance with another embodiment of the present technology.

FIG. 3A shows a top perspective view of an illustrative embodiment of a device for culturing cells in accordance with an embodiment of the present technology.

FIG. 3B shows a cross-sectional side view of an illustrative embodiment of the device shown in FIG. 3A.

FIG. 4 shows transfer by pipette of at least one test cell and a culture medium to at least one cell culture well in an illustrative embodiment of the device for culturing cells shown in FIG. 3A and FIG. 3B.

FIG. 5 shows a micrograph of an illustrative embodiment of a fibroblast cell culture growing in at least one cell culture well in accordance with an embodiment of the present technology.

FIG. 6 shows an illustrative embodiment of a device for culturing cells including at least one sensor in proximity to at least one cell culture well, a controller in communication with the at least one sensor, and at least one fluid flow regulator in communication with the controller, in accordance with an embodiment of the present technology.

FIG. 7 shows a schematic diagram of an embodiment of a microfluidic chip.

FIG. 8 shows a micrograph of fibroblasts in an exemplary microwell.

FIG. 9 shows a micrograph of HeLa cells in an exemplary microwell.

FIG. 10A shows a micrograph of Human Umbilical Vein Endothelial Cells (HUVEC) in an exemplary microwell.

FIG. 10B shows a micrograph of HUVEC on a Petri dish.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present technology bridges the gap between traditional static cell culture techniques and dynamic cell culturing in microfluidic devices by providing a patterned, multilayer microfluidic device that includes at least one cell culture well approximately 2-3 mm in diameter. As such, the array of microwells may accommodate larger cells such as embryonic cells, for example. Further, access to the cell culture is available by way of a retractable top layer. The retractable top layer effectively seals the cell culture in a controllable environment.

Cells may be delivered to the at least one cell culture well through pipettes using conventional methods. After loading the cells, culture media may be delivered through the microchannels. The microchannels may be used to feed and drain the at least one cell culture well. By doing so, the culture media remains fresh, and the concentration and relative speed of the culture media may be monitored to vary and determine cell growth, for example.

The microfluidic device may be composed of an elastomer, as an example polydimethylsiloxane (PDMA). The microfluidic patterned multilayer device may include, in addition to the at least one cell culture well, microvalves and micropumps. The microfluidic device microvalves and micropumps may be controlled pneumatically or by a syringe, where an externally accessible port in fluid communication with at least one of the at least one microfluidic channels may accommodate a variety of hypodermic needles ranging from #6-#36 gauge, for example.

The microfluidic devices that include the at least one cell culture well may be scaled up in both dimension and number. An array of microwells may include one, two, and three dimensional arrays. The array of microwells may include a mechanism to transfer serially a cell cultured in a first microwell to a second microwell, where the first and second microwell may provide disparate growing environments as governed by one of: a fluidic or aqueous cell culture medium and an atmospheric medium.

### Device(s) of the Present Technology Useful for Cell Culture

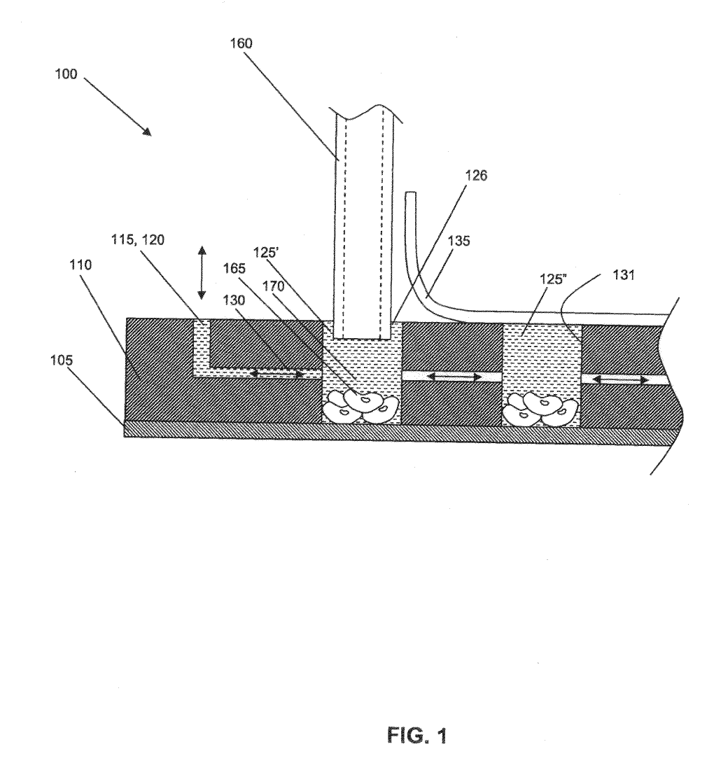

FIG. 1 shows a side view of an embodiment of a device for culturing cells 100 including a backing layer 105 and one interconnecting layer 110 in accordance with an embodiment of the present technology. In some instances, this can be referred to as a microfluidic chip. The interconnecting layer 110 includes a pattern defined therein. The pattern includes at least one microfluidic channel 130. The pattern may also include at least one cell culture well 125′, 125″ (generally 125), each in fluid communication with at least one of the microfluidic channels 130. The cell culture well 125 has an opening at one end 126 and a side wall 131, the at least one microfluidic channel 130 in fluid communication with the side wall of the at least one cell culture well 125. The microfluidic channels 130 each have a maximum channel width that is substantially less than a maximum width of any of the cell culture wells 125.

The backing layer 105 generally provides a support for the one or more interconnecting layers 110. The backing layer 105 can be flexible, semi-rigid, or rigid, depending upon the intended application. In some embodiments, the backing layer 105 is formed from a crystal or glass. The backing layer 105 can be substantially planar, such as plate glass or semiconductor wafer substrates. In some embodiments, the backing layer 105 can be non-planar. For example, the backing layer can be cylindrical, with the interconnecting layer formed thereupon along one or more of an inner and outer surface of the cylinder.

In some embodiments, the maximum width of the cell culture well 125 is at least ten times greater than the maximum microfluidic channel 130 width. In some embodiments, the maximum microfluidic channel 130 width of the at least one cell culture well 125 is at least sixty times greater than the maximum microfluidic channel 130 width. In some embodiments, the one interconnecting layer 110 of the device for culturing cells 100 includes polydimethylsiloxane (PDMS).

In some embodiments, the device for culturing cells 100 further includes one or more externally accessible ports 115, 120, each in fluid communication with at least one of the microfluidic channels 130. In some embodiments, the device 100 further includes a top layer 135 adapted to cover each of the at least one cell culture well 125. The top layer 135 can be removable, selectively allowing access to open ends 126 of one or more of the cell culture wells 125. The top layer 125 may be rigid, semi-rigid, or flexible, depending upon the intended application. In some embodiments, the removable top layer 135 can be translucent, allowing at least some light to pass through, or transparent providing a window into the one or more cell culture wells 125. The removable top layer 135 can include one of: PDMS, polymethyl methacrylate (PMMA), and a glass.

In some embodiments, the at least one cell culture well 125 contains at least one test cell 165 and a culture medium 170. In some embodiments, the test cell 165 is a pluripotent cell. In some embodiments, the test cell 165 and the culture medium 170 are transferred to and from the at least one cell culture well 125 by pipette 160. In some embodiments, the removable top layer 135 provides an operator access to the test cell 165 and the culture medium 170 in the at least one cell culture well 125.

At least one advantage of the device 100 is that it allows for transfer of the test cell 165 and the culture medium 170 by pipette 160 without requiring that any of the test cells 165 flow through any of the microfluidic channels 130. Removing a step wherein the test cell 165 flows through the least one microfluidic channel 130 reduces shear and compressive forces to the test cell 165, which may be damaging to a fragile test cell 165. Further, flowing a culture medium 170 through the at least one microfluidic channel 130 to the side wall of the at least one cell culture well 125 reduces turbulence effects on the test cell 165 and reduces the chance of blocking the at least one microfluidic channel 130 with the test cell 165 itself, as the test cell 165 may be positioned above or below a side wall entry point of the at least one microfluidic channel 130. Also, the size of the test cells 165 is not restricted by dimensions of the microfluidic channels 130.

In some embodiments, the test cell 165 may be transferred to adhere to a side or the bottom of the at least one cell culture well 125. In some embodiments, the test cell 165 may be transferred to be submerged in the culture medium 170 within the cell culture well 125. In some embodiments, the test cell 165 may be transferred to float at or near the top of the culture medium 170 within the cell culture well 125. In some embodiments, the test cell 165 position may be controlled within the cell culture well 125 during the transferring process by one or more of: an electrostatic charge, a magnetostatic moment, a chemical binding mechanism, and a surface adherence to the walls of the cell culture well 125.

In some embodiments, following the transferring of the test cell 165 and the culture medium 170 to the at least one cell culture well 125, the removable top layer 135 is reversibly sealed over the at least one cell culture well 125. The seal effectively isolates the environment of the test cell 165 and the culture medium 170 by isolating the cell culture well 125 from an external environment.

The one or more interconnecting layers 110 of the device for culturing cells 100 may further include a removable top layer 135 adapted to cover each of the at least one cell culture well 125. In some embodiments, the removable top layer 135 is transparent. The removable top layer 135 includes one of: PDMS, polymethyl methacrylate (PMMA), and a glass. The removable top layer 135 provides an operator with physical access to a test cell 165 and a culture medium 170 in the at least one cell culture well 125; whereas, a transparent top layer 135 provides an operator with visual access.

In some embodiments, the environment of the test cell 165 may be monitored and may be controlled. For example, in some embodiments, monitoring the test cell 165 may include one or more of: measuring an at least one microfluidic channel 130 fluid velocity, measuring an at least one microfluidic channel 130 fluid ionic composition, measuring an at least one cell culture well 125 temperature, measuring an at least one cell culture well 125 pressure, measuring a test cell 165 and a culture medium 170 optical transmissivity, optical reflectivity, and spectroscopic data.

In some embodiments, measuring the fluid ionic composition in the at least one cell culture wells 125 may include measuring using a capillary electrophoresis (CE) with a laser-induced fluorescence (LIF) detector. In some embodiments, measuring spectroscopic data may include measuring one or more of: an optical spectrum, an infrared spectrum (IR), an Fourier Transform infrared spectrum (FTIR), and a nuclear magnetic resonance (NMR) spectrum of the test cell 165. In some embodiments, optical spectrum data is accessed through the transparent removable top layer 135. In some embodiments, IR and FTIR spectrum data are accessed through the transparent removable top layer 135 by further requiring the removable top layer 135 to be transparent with respect to a portion of the IR spectrum. NMR data can be recovered in the absence of paramagnetic and ferromagnetic materials in the one or more interconnecting layers 110 of the device for culturing cell 100.

In some embodiments, the device 100 includes an environmental cavity control for controlling one or more of: a temperature, a pressure, a partial pressure, and a chemical environment within on or more of the cell culture wells 125. In an exemplary chemical control of the environmental cavity of the at least one cell culture well 125, the chemical environment is an oxygen (O₂) environment. For example, temperature may be controlled through an external thermal device, such as a heater (e.g., resistive heater, exothermic/endothermic chemical reaction, thermoelectric cooler/heater, and any suitable heat exchanger, such as a heat sink). The chemical environment can be controlled by the introduction of one or more chemicals or compounds into the cell culture wells 125. Such chemicals or compounds can be introduced and/or removed through one or more of the microchannels 130 and the open end 126 of the cell culture well 125.

In a further embodiment to sealing the removable top layer 135 and controlling a chemical environment, a reactive catalyst, such as platinum (Pt) for example, may be affixed to the underside of the removable top layer 135 to encourage a chemical reaction between one or more of: an unsubmerged test cell 165 and a surface of the culture medium 170 in the at least one cell culture well 125.

In a further embodiment to sealing the removable top layer 135 and controlling a chemical environment, a radiative reactive catalyst, UV radiation for example, can be directed into the at least one cell culture well 125 through one or more of the microchannels 130 and the transparent removable top layer 135 to encourage a chemical reaction between one or more of an unsubmerged test cell 165 and a surface of the culture medium 170 in the at least one cell culture well 125. Two examples of a cellular reaction dependent on UV radiation as a catalyst are the evolution of Vitamin D in animal cellular chemistry and photosynthesis in plant cellular chemistry. In some embodiments, such a radiative reactive catalyst can be directed into the at least one cell culture well 125 through one or more of a bottom layer and one or more of the interconnecting layers.

Following an experiment or group of experiments, test cells 165 one or more of the cell culture wells 125 may be harvested. In some embodiments, harvesting includes peeling back the removable top layer 135, removing one or more of the test cells 165, by pipette 160 for example. The one or more test cells 165 may be transferred to a secondary experimental or analysis station. The removable top layer 135 can be reversibly replaced, thereby resealing the one or more cell culture wells 125.

As described herein, the one or more interconnecting layers 110, can be configured to define one or more of: the cell culture wells 125; the at least one microfluidic channel 130; the one or more externally accessible ports 115, 120; and for that matter, any other pattern therein. Such structures 125, 130, 115, 120 can be formed by techniques known to those skilled in the art, such as molding, embossing, laser drilling and laser ablation, conventional drilling, soft lithography, and porous laminates.

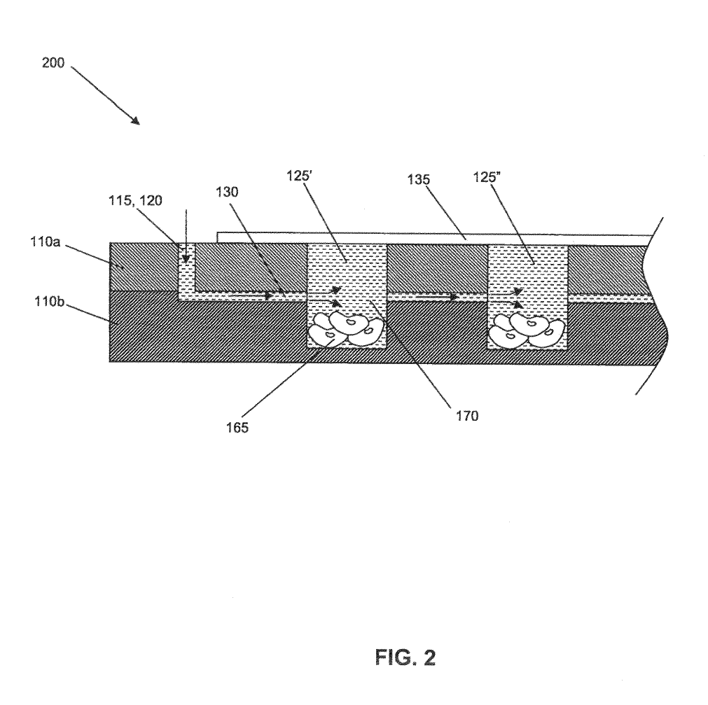

FIG. 2 shows a cross-sectional side view of an embodiment of a device for culturing cells 200 including two interconnecting layers 110a, 110b. FIG. 2 demonstrates the at least one microfluidic channel 130 formed by both of the two interconnecting layers 110a, 110b, For example, an open channel (i.e., a trough) can be formed in one of the interconnecting layers 110a, 110b, whereby the other connecting layer when positioned to abut an elongated opening of the trough, forms a lumen of the microchannel 130. In other embodiments, a complementary open channel (i.e., a trough) can be formed in each of the interconnecting layers 110a, 110b, whereby the properly aligned connecting layers positioned to abut each other form a lumen of the microchannel 130. A possible advantage in forming the at least one microfluidic channel 130 by both of the two interconnecting layers 110a, 110b is in ease of construction.

The at least one microfluidic channel 130 formed by both of the two interconnecting layers 110a, 110b allows for a soft lithography to pattern a first portion of the at least one microfluidic channel 130 in a first interconnecting layer 110a and to pattern a second portion of the at least one microfluidic channel 130 in a second interconnecting layer 110b, obviating the need for means known to those skilled in the art, such as molding, embossing, laser drilling and laser ablation, conventional drilling, and porous laminates.

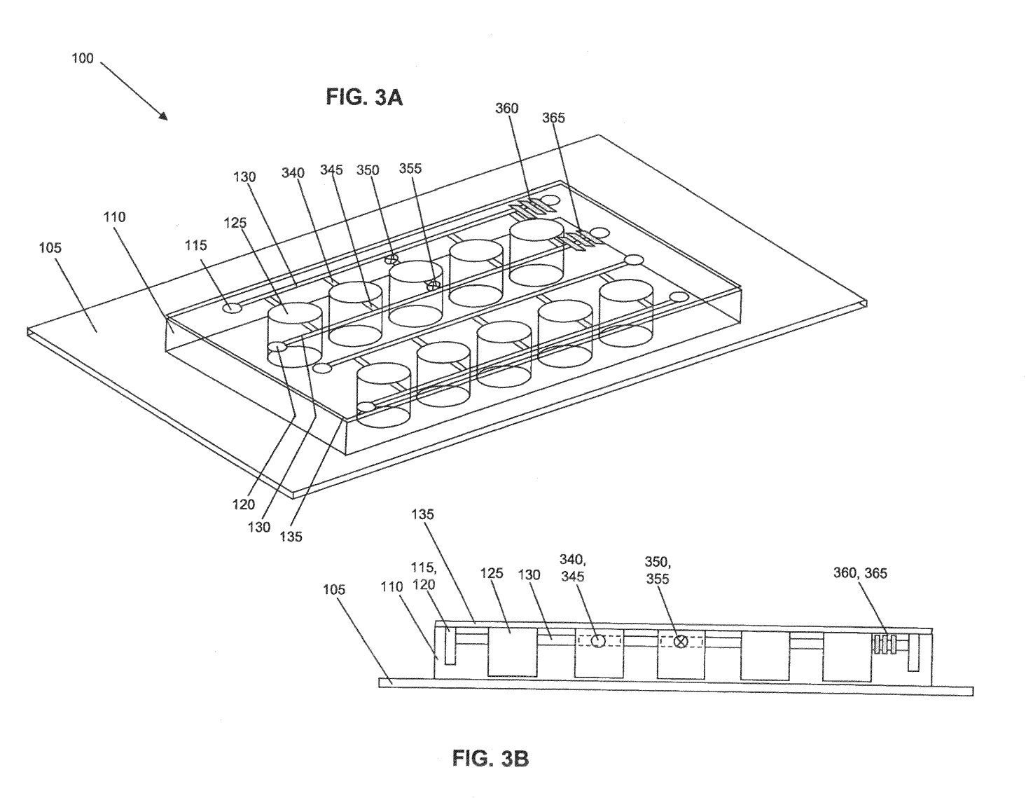

FIG. 3A shows a top perspective view of an embodiment of the device for culturing cells 100 including multiple interconnecting layers 110 in accordance with an embodiment of the present technology. FIG. 3B shows cross-sectional a side view of the device 100 including multiple interconnecting layers 110 in accordance with an embodiment of the present technology.

In some embodiments, the device for culturing cells 100 further includes an at least one controllable valve 350, 355. For example, the controllable valves 350, 355 can be housed in an at least one controllable valve channel 340, 345 in fluid communication with at least one of the microfluidic channels 130. One or more of the controllable valves 350, 355 can be configured to selectively restrict fluid transport through at least one of the microfluidic channels 130 and the respective controllable valve channel 340, 345. In some embodiments, one or more of the controllable valves 350, 355 can be a flexure valve.

Alternatively or in addition, the device for culturing cells 100 includes one or more pumps 360, 365 positioned in fluid communication with at least one of the microfluidic channels 130. Each of the one or more pumps 360, 365 is configured to transport fluid through at least one of the microfluidic channels 130. In some embodiments, one or more of the pumps 360, 365 can be peristaltic pumps.

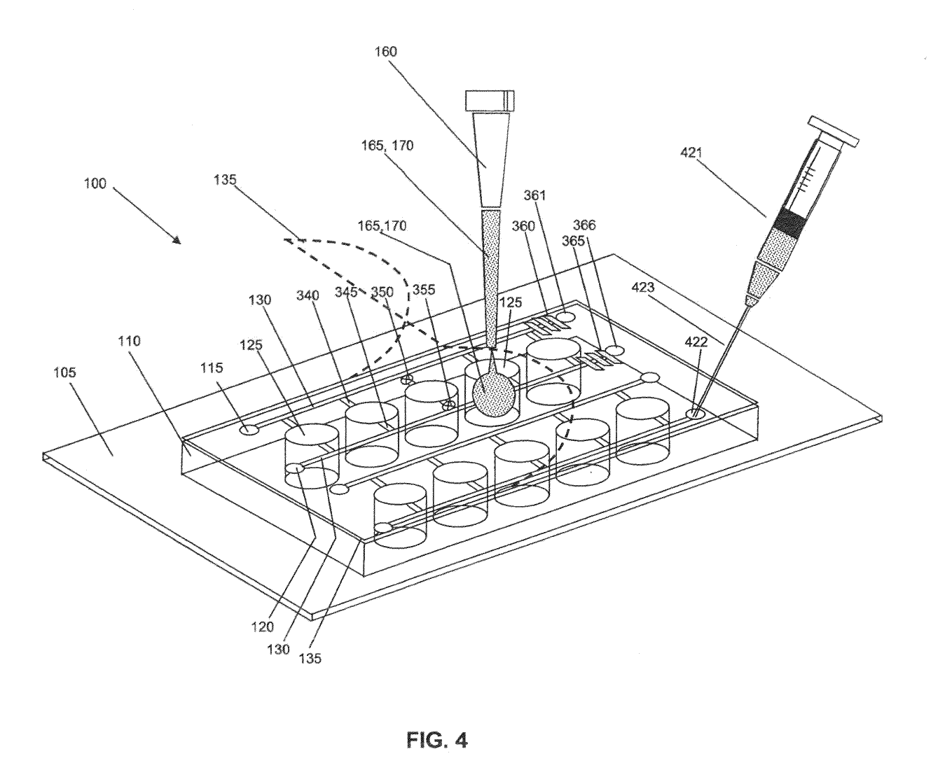

FIG. 4 shows transferring by pipette 160 at least one of a test cell 165 and a culture medium 170 to one or more of the cell culture wells 125 in the device for culturing cells 100 in accordance with an embodiment of the present technology. In some embodiments, a mechanical pump, such as a syringe 421 is used to transfer one or more of the test cells 165 and culture medium 170 through the externally accessible port 422, which is in fluid communication with at least one of the microfluidic channels 130. The externally accessible port 422 may be formed by a lumen extending from at least one of the microfluidic channels 130 to an external surface of the device 100. For example, the externally accessible port 422 may be selectively accessible from a top surface of the device 100, being positioned under the removable top 135.

The externally accessible port 422 can have a diameter equal to, or substantially different than the one or more interconnected microfluidic channels 130. For example, the externally accessible port 422 can be cylindrical, having a diameter of between about 0.1 mm and about 5.0 mm, whereas the microfluidic channels 130 may have diameters that are substantially smaller. In some embodiments, the diameter of the externally accessible port 422 allows access to the externally accessible port 422 by a hypodermic needle 423, where the approximate diameter of the externally accessible port 422 of 0.1-5.0 mm approximate the outside diameters (OD) of a #36-#6 gauge hypodermic needle, for example. The cylindrical externally accessible port 422 can extend to a depth of the at least one microfluidic channels 130, or deeper. For example, the externally accessible port 422 can extend into the interconnecting layer to a depth of approximately 2-3 mm. In some embodiments, the externally accessible port 422 is conical, transitioning from a first diameter at an outside surface of the device 100 to a different (e.g., smaller) diameter at a depth measured relative to the outside surface.

In some embodiments, each of the cell culture wells 125 can be cylindrical in shape, extending from the open end to a bottom surface of the well. The cylindrical shape may be a right cylinder, or a slant cylinder. The cross sectional shape of the cylinder may be circular, elliptical, polygonal, or irregular. In some embodiments, all of the cell culture wells 125 are substantially similar in shape in size. Alternatively, at least some of the cell culture wells 125 can differ with respect to each other in one or more of size and shape. The bottom of the cell culture well 125 may be planar, or non planar.

Generally, the cell culture wells 125 are dimensioned to accept a test cell size 165 and volume of culture medium 170. For example, the cell culture wells 125 may be right cylinders, approximately 2-3 min in diameter and extend approximately 4-6 mm in depth. In some embodiments, the at least one microfluidic channel 130 may have a cross section of approximately 100 microns in width and approximately 10 microns in depth.

One or more of the cell culture wells 125 are in fluid communication with one or more of the microfluidic channels 130. One or more of the microfluidic channels 130 may intersect a respective one of the cell culture wells 125 along a top surface, a bottom surface, or a side surface of the cell culture well 125. For embodiments in which a microfluidic channel 130 intersects a side surface, the point of intersection may be disposed close to one of the top or bottom surfaces, or at any position therebetween. Thus, a cell culture well 125 storing a test cell 165 disposed along a bottom portion of the well 125 as shown in FIGS. 1 and 2, may intersect with at least one microfluidic channel 130 at a point along a side wall positioned substantially above the test cell 165. Thus, fluids may be transferred into and or out of the cell culture well 125 without blockage by the test cell 165, and preferably minimizing any shear forces resulting from the fluid flow on the test cell 165.

In some embodiments, the at least one controllable valve 350, 355 housed in the at least one controllable valve channel 340, 345 in fluid communication with the at least one microfluidic channel 130, may be activated pneumatically according to techniques well known to those skilled in the art. For example, an applied negative pressure may drive the at least one controllable valve 350 open to gate a culture medium 170 from the at least one microfluidic channel 130 to the at least one cell culture well 125. An applied positive pressure may drive the at least one controllable valve 350 closed to inhibit a culture medium 170 from flowing from the at least one microfluidic channel 130 to the at least one cell culture well 125.

Similarly, an applied negative pressure may drive the at least one controllable valve 355 open to gate a culture medium 170 from the at least one cell culture well 125 to the at least one microfluidic channel 130. An applied positive pressure may drive the at least one controllable valve 355 closed to inhibit a culture medium 170 from flowing from the at least one cell culture well 125 to the at least one microfluidic channel 130.

As an alternative embodiment to activating the at least one controllable valve 350, 355 pneumatically, the at least one controllable valve 350, 355 may be activated by a positive microfluidic partial pressure in the at least one microfluidic channel 130. A positive partial pressure may force open the at least one controllable valve 350, 355, gating a culture medium 170 into the at least one cell culture wells 125.

As an alternative embodiment to activating the at least one controllable valve 350, 355 pneumatically, the inner diameter (ID) of the at least one controllable valve 350, 355 may be made sufficiently small such that diffusion into and out of the at least one cell culture wells 125 is inhibited by a surface tension at or about the surfaces of the at least one controllable valve 350, 355.

In each of the aforementioned embodiments addressing the at least one controllable valve 350, 355, a differential partial pressure may be required to drive a culture medium 170 from the at least one microfluidic channel 130 to the at least one cell culture well 125 and a differential partial pressure may be required to drive a culture medium 170 from the at least one cell culture well 125 to the at least one microfluidic channel 130.

In some embodiments, the differential partial pressure may be provided by injecting a culture medium 170 directly into the externally accessible port 115 by a hypodermic needle 423, for example, and by removing a culture medium 170 directly from the externally accessible port 120 by a hypodermic needle 423, for example, or by a vacuum pump, for example. As previously mentioned, the diameter of the externally accessible port 115, 120, 422 allows access to the ports by a hypodermic needle 423, where the diameter of the externally accessible port 115, 116, 422 approximate the outside diameters (OD) of a #36-#6 gauge hypodermic needle 423, for example.

In an alternative embodiment, the differential partial pressure may be provided by at least one peristaltic pump 360, 365 that may reside in the at least one microfluidic channel 130. In some embodiments, the at least one peristaltic pump 360, 365 separate the externally accessible port 361, 366 and the at least one microfluidic channel 130.

The at least one peristaltic pump 360, 365 may increase a pressure in the at least one microfluidic channel 130 with respect to the pressure in the at least one cell culture well 125, driving a culture medium 170 into the at least one cell culture well 125 through the at least one controllable valve 350, 355 housed in the at least one controllable valve channel 340, 345.

The at least one peristaltic pump 360, 365 may decrease a pressure in the at least one microfluidic channel 130 with respect to the pressure in the at least one cell culture well 125, removing a culture medium 170 from the at least one cell culture well 125 through the at least one controllable valve 350, 355 housed in the at least one controllable valve channel 340, 345. In some embodiments, the at least one peristaltic pump 360, 365 may include one or more serially activated pneumatic flexure microvalves.

The one or more interconnecting layers 110 of the device for culturing cells 100 may include an elastomer. Elastomers are collections of long polymeric chains including, but not limited to, carbon, hydrogen, oxygen, and/or silicon. Elastomers existing above their glass temperature (T_{g}) are amorphous polymers, where considerable segmental motion of individual chains is possible, giving the elastomer properties of a fluid.

Elastomers can be cured by heating in the presence of a curing agent. Curing refers to the process of cross linking. Cross links are covalent bonds linking one polymer chain to another. Cross linking is the characteristic property of thermosetting plastic materials. Cross linking inhibits close packing of the polymer chains, preventing the formation of crystalline regions. The restricted molecular mobility of a cross linked structure limits the extension of the polymer material under loading.

Cross links are formed by chemical reactions that are initiated buy heat and/or pressure, or by the mixing of an unpolymerized or partially polymerized resin with various chemicals; cross linking can be induced in materials that are normally thermoplastic through exposure to radiation such as, but not limited to, ultraviolet (UV) radiation, infrared (IR) radiation, and electromagnetic (EM) radiation. Across linked elastomer can reversibly extend to between 5% and 700% in length, with no macroscopic distortion.

Many unsaturated elastomers, such as naturally occurring rubber, are cured in the presence of sulfur, a process call vulcanization. Elastomers cured by vulcanization include, but are not limited to: natural rubber, polyisoprene, butyl rubber, halogenated butyl rubbers, polybutadiene, styrene butadiene, nitrile rubber, hydrated nitrile rubbers, and chloroprene rubbers such as polychloroprene, Neoprene, and Baypren.

Saturated elastomers cannot be cured by vulcanization. Elastomers that cannot be cured by vulcanization include, but are not limited to: ethylene propylene rubber, epichlorohydrin rubber, polyacrylic rubber, silicone rubbers, fluorosilicone rubber, fluoroelastomers, and in generally any synthetic rubber, such as VITON, a registered trademark of E. I. Du Pont de Nemours & Company, and any fluoroelastomers (FKM) and perfluoroelastomers (FFKM), such as TECNOFLON, a registered trademark of Solvay Solexis S.p.A., of Italy, and perfluoroelastomers, tetrafluoro ethylene/propylene rubbers, chlorosulfonated polyethylene, and ethylene vinyl acetate.

Elastomers that fall into neither category of saturated nor unsaturated elastomers include, but are not limited to: thermoplastic elastomers, polyurethane, resilin, elastin, polyimides, phenol formaldehyde polymers, and polydimethylsiloxane (PDMS). Elastomers in this category have lower glass temperatures (T_{g}), generally much below room temperature, and as a result, possess the properties of a fluid at or near room temperature.

PDMS, for example, has many of the material properties favorable to microcasting, micromolding, and micropatterning. The glass temperature for PDMS is very low, T_{g}=−120 C. As a result, the viscosity for PDMS is relatively low at room temperature, approximating the viscosity, of honey, where is approximately equal to 1750 cP. This allows PDMS to flow in a master with properties of a fluid.

PDMS is cured at temperatures ranging from room temperature to 80 C. PDMS is cured with the addition of a set of curing agents including at least one of the following: a platinum catalyst complex, copolymers of methyhydrosiloxane, and copolymers of dimethylsiloxane. Curing occurs by hydrosilylation, cross linking between vinyl terminated PDMS groups (SiCH═CH₂) and hydrosilane (SiH) groups. The precursor, in this example PDMS, is hardened by a curing process. Cured PDMS has a viscosity of about 5.1+/−0.9×10⁷ cP, which gives cured PDMS a viscosity approximately between the working point (n=10⁶ cP) and the softening point (n=10^{9.5} cP) of glass.

Solvents can be used to reduce the viscosity of the precursor. Exemplary solvents for PDMS include methanol, glycerol, and water. Solvents such as methylene chloride, acyclic and cyclic hydrocarbons, aromatic hydrocarbons, halogenated compounds, ethers, and amines can possibly cause damage when producing fine feature microfluidic channels, as this set of solvents diffuse into PDMS and swell the PDMS precursor, damaging fine features in the master and effectively closing off microfluidic channels in the interconnected multilayer patterns. Solvents such as acetone, 1-propanol, and pyridine can swell PDMS to a lesser extent.

The precursor, or the precursor and solvent, must be degassed prior to curing. Any remaining gas particles dissolved and entrapped ion the precursor can easily find their way to the master, adhering to critical features, creating voids in the fabricated microfluidic channels. Degassing can be performed by placing the entire master, precursor, and rigid substrate backing layer inside a vacuum chamber. The vacuum chamber should be set at a pressure no greater than 20-25 mm of mercury. Degassing of PDMS can take from 30 minutes to two hours, depending on the pattern density and geometry size of the master. Some air bubbles may remain entrapped, adhering to the master surface, but will burst upon back filling of the air into the vacuum chamber.

Cleaning a master between serial microfluidic channel molds can lengthen the lifetime of the master, allowing the master to be used to fabricate up to fifty or more PDMS microfluidic channel patterns. Solvents used to clean the master may include, but are not limited to, methanol, glycerol, and water, the same solvents used to reduce the viscosity of the precursor. Residual solvent, solvent that is not evaporated following a cleaning step, can diffuse into the next PDMS precursor application. Chosen incorrectly, the residual solvent following a cleaning step can swell the PDMS volumetrically precisely at the finest master feature sites, as described previously for solvents used to reduce the viscosity of the precursor.

Feature sizes associated with an elastomeric microfluidic channel pattern such as the one or more interconnecting layers 110 of the microfluidic device 100 in the aforementioned preferred embodiments can be as small as approximately 30 nm, which is roughly 60% of the feature size limit associated with standard photo lithography. Feature size aspect ratios can be as high as approximately 2.0 or higher with very little feature distortion.

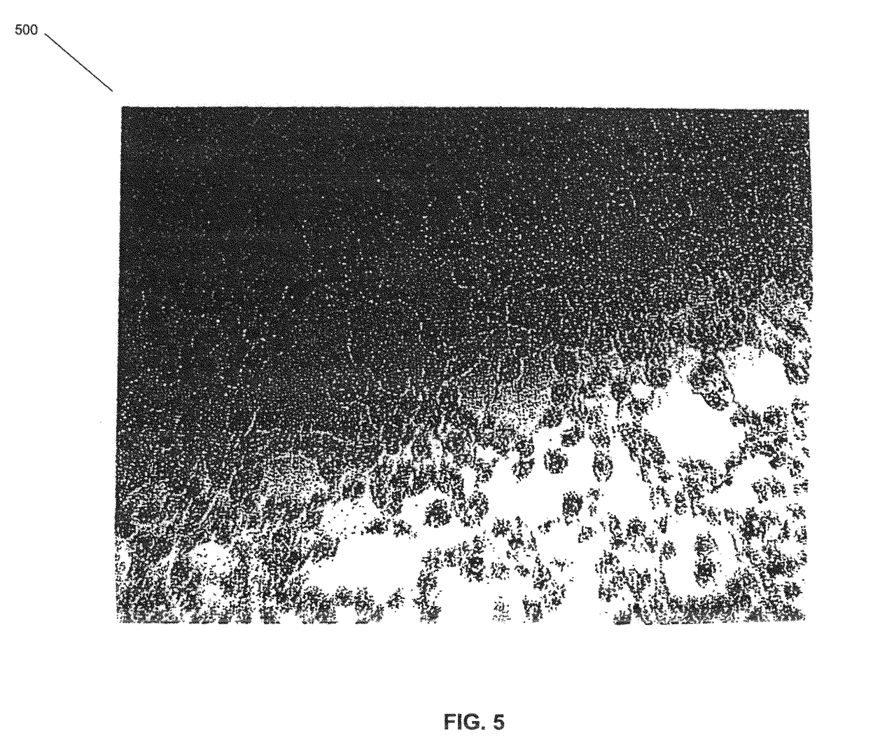

FIG. 5 shows a micrograph of a fibroblast cell culture 500 growing in at least one cell culture well 125 in accordance with an embodiment of the present technology. The micrograph image was taken in situ, prior to a harvesting step of the test cell 165, and is provided for and made possible by the transparency of the removable top layer 135.

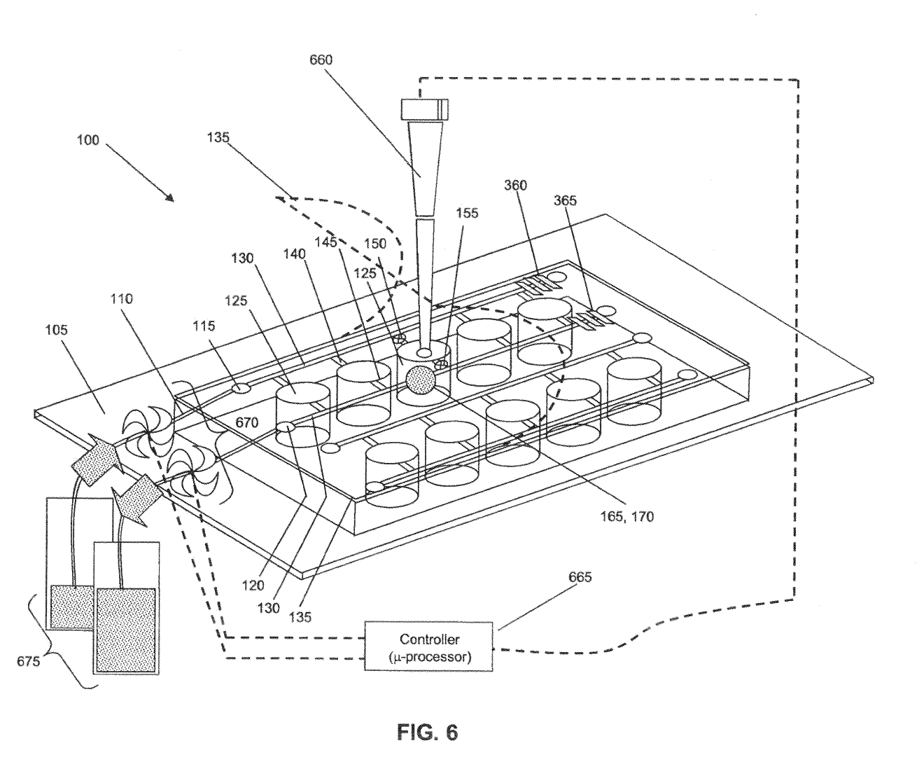

FIG. 6 shows an alternative system configuration of the device for culturing cells 100 including at least one sensor 660 in proximity to at least one cell culture well 125. The system includes a controller 665 in communication with the at least one sensor 660, and at least one fluid flow regulator 670 in communication with the controller 665. In some embodiments, at least one sensor 660 is configured to observe a physical attribute of a set of test cells 165 and culture medium 170 disposed within the at least one cell culture well 125.

The physical attributes of the test cells and culture medium 170 can include temperature, pressure or partial pressure, chemical composition, luminosity, color, clarity, size, shape, number, weight. Accordingly, one more suitable sensors are selected depending upon the particular physical attribute(s) to be measured. Sensors can be selected from the group including an image sensor, a flow rate sensor, an ionic composition sensor, a temperature sensor, a pressure sensor, a light sensor, and a spectroscopic sensor.

Image sensors include charge coupled devices (CCD) of other suitable sensor to obtain an electronic image of the test cells 165 and culture medium 170 within the cell culture well 125. Light sensors include photodiodes, avalanche photodiodes, and phototransistors configured to detect one or more of light emitted by, transmissivity of light shone through, and reflectivity of light from the test cells 165 and/or culture medium 170. Temperature sensors include thermocouples and thermometers. Pressure sensors include barometers and stress or strain gauges.

In some embodiments, the controller 665 is configure to adjust at least one of the fluid flow regulators 670, thereby regulating transportation of fluid through the at least one microfluidic channel 130 responsive to the one or more physical attributes observed by the at least one sensor 660. In some embodiments, the control 665 is a microprocessor. The controller 665 can include one or more of software, hardware, and firmware to process instructions responsive to data received from the at least one sensor 660. In some embodiments, the data processing includes image processing of an image data from an image sensor. The image may be a magnified image as may be obtained from a microscope. The controller 665 adjusts environment of one or more of the cell culture wells 125 by regulation of fluid flow through the one or more microfluidic channels 130. For example, a volume and/or rate of nutrients supplied through a flow of culture medium 170 to a cell culture well 125 housing the test culture cell 165 can be varied by the controller 665 according to a size and/or weight of the test cell 165 determined by the sensor. Thus, the system is able to run in a closed loop manner varying environment of the cell culture wells 125 according to feedback from the sensors monitoring the test cells 165 and culture medium 170 within the wells 125.

One or more or any part thereof of the measurement, feedback, and/or spectroscopic measurement, recording, and display techniques described above can be implemented in computer hardware or software, or a combination of both. The process can be implemented in computer programs using standard programming techniques following the process and figures described herein. Program code is applied to input data to perform the functions described herein and generate output information. The output information is applied to one or more output devices such as a display monitor. Each program may be implemented in a high level procedural or object oriented programming language to communicate with a computer system. However, the programs can be implemented in assembly or machine language, if desired. In any case, the language can be a compiled or interpreted language. Moreover, the program can run on dedicated integrated circuits preprogrammed for that purpose.

Each such computer program is preferably stored on a storage medium or device (e.g., ROM or magnetic diskette) readable by a general or special purpose programmable computer, for configuring and operating the computer when the storage media or device is read by the computer to perform the procedures described herein. The computer program can also reside in cache or main memory during program execution. The analysis process can also be implemented as a computer-readable storage medium, configured with a computer program, where the storage medium so configured causes a computer to operate in a specific and predefined manner to perform the functions described herein.

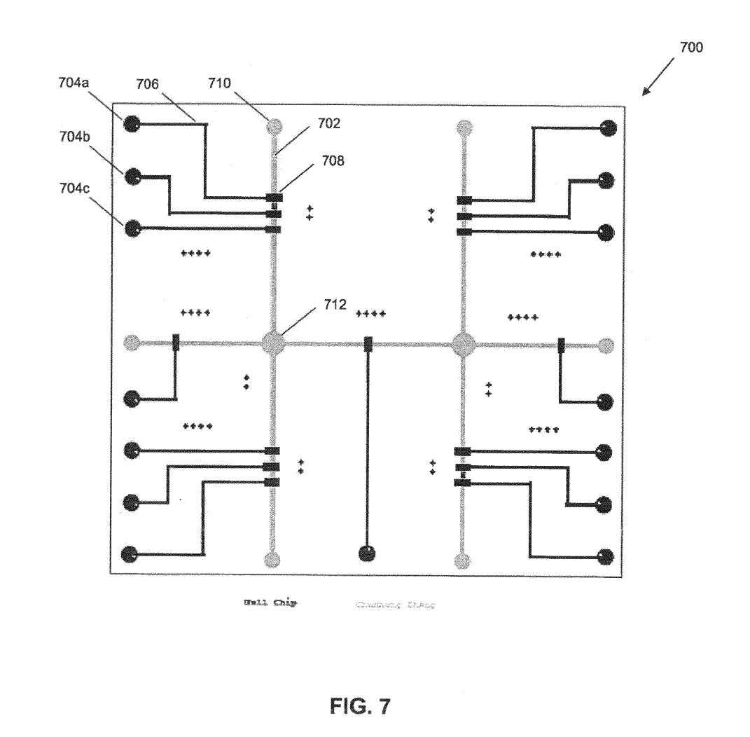

A schematic diagram of an exemplary embodiment of a microfluidic chip 700 is shown in FIG. 7. The microfluidic chip 700 includes a planar arrangement of cell culture wells 704a, 704b, 704c, . . . (generally 704). At least some of the cell culture wells 704 include an opening at one end providing direct access to the interior volume of the cell culture well 704. The microfluidic chip 700 also includes one or more microfluidic distribution channels 702. The exemplary device 700 includes a rectilinear arrangement of such microfluidic distribution channels 702. At least one of the microfluidic distribution channels 702 is in fluid communication with an externally accessible port 710. The externally accessible port 710 can be used to insert and/or extract fluid from the device 700, for example using a syringe or pump or any other suitable pumping method described herein.

Each of the cell culture wells 704 is in fluid communication with one of the microfluidic distribution channels 702 through a respective microfluidic channel 706. At least some of the microfluidic channels 706 intersect sidewalls of the cell culture wells 704 as described herein. A junction 708 formed at the intersection of the microfluidic channel 706 with the microfluidic distribution channel 702 can be an unrestricted fluid junction. Alternatively, the junction 708 can include a controllable fluid flow element 708, such as a controllable microfluidic valve. Similarly, one or more of the junctions 712 formed at the intersection of two or more microfluidic distribution channels 702 can also be an unrestricted fluid junction. Alternatively, the junction 712 can include a controllable fluid flow element 708, such as a microfluidic valve. One or more of such valves 708, 712 can be controlled using well established techniques for controlling fluid flow within a microfluidic device. Generally, the dimensions of the cell culture well 704 are substantially larger than cross-sectional dimensions of the microfluidic channels 706. In the exemplary embodiments, the radius of each cell culture well 704 is about 1.5 mm, with each cell culture well 704 sized to contain at least about 30 μL.

In operation, cell cultures can be inserted into one or more of the cell culture wells 704 as described herein through an open access port. Fluids, such as nutrients can be selectively channeled into one or more of the cell culture wells 704 and wastes selectively channeled out, to provide a controllable environment to the cell cultures. Fluid can be injected into or removed from the device 700 through the one or more externally accessible ports 710. In some embodiments, the arrangements of cell culture wells 704 can

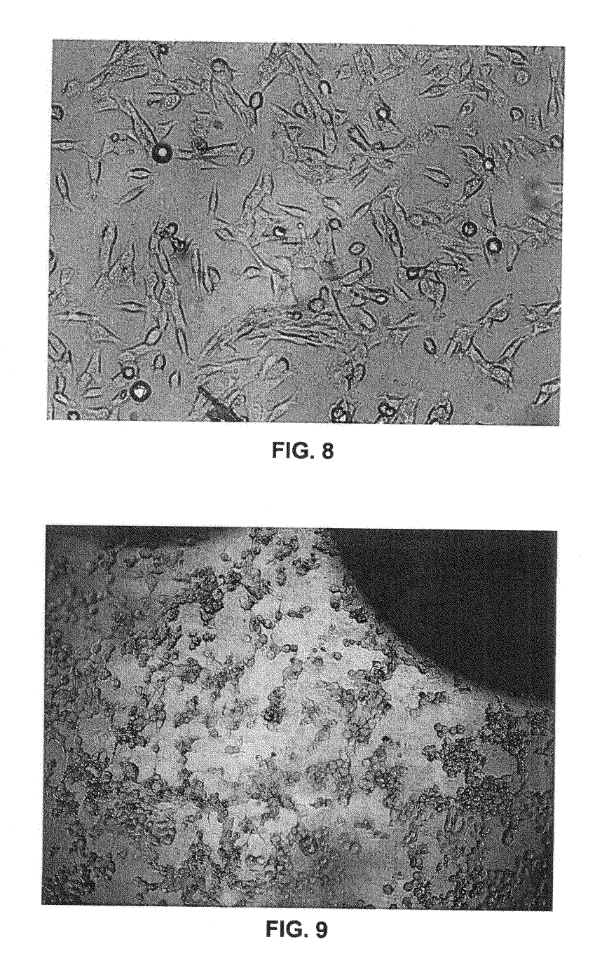

A first test case relates to culturing fibroblast cells. Microwells were incubated with 50 μg/mL fibronectin for one hour prior to seeding with fibroblasts. Fibroblasts were added in 10 μL medium. The cells were incubated at 37° C. with 5% CO₂. FIG. 8 is a micrograph of fibroblasts after four hours of growth in the microwell.

A second test case relates to culturing HeLa cells. Microwells were incubated with Collagen for one hour. HeLa cells were added in 10 μL medium. The cells were incubated at 37° C. with 5% CO₂. FIG. 9 is a micrograph of the HeLa cells after ten hours of growth in the microwell.

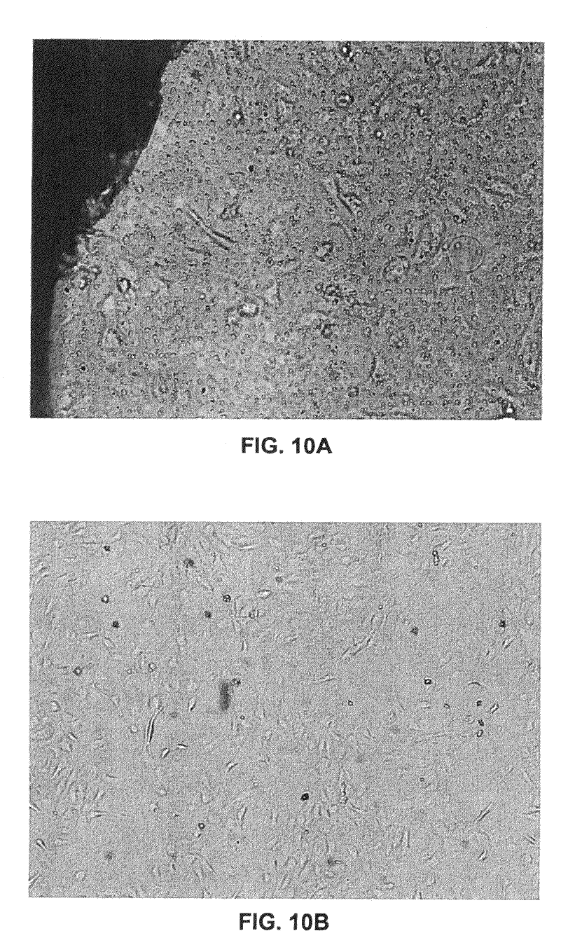

A third test case relates to culturing endothelial cells. Microwells were incubated with 50 μg/mL fibronectin for one hour. Human Umbilical Vein Endothelial Cells (HUVECs) were added in 10 μL medium. The cells were incubated at 37° C. with 5% CO₂. FIG. 10A is a micrograph showing HUVEC growth after two hours in a microwell. FIG. 10B is a micrograph showing HUVEC after two hours of growth on a Petri dish under substantially the same growth conditions as used for the microwell shown in FIG. 10A.

A number of embodiments of the technology have been described. Nevertheless, it will be understood that various modifications may be made without departing from the spirit and scope of the technology.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### EQUIVALENTS

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. An apparatus for culturing cells, comprising:
one or more interconnecting layers having a pattern therein, the pattern comprising at least one microfluidic channel;
at least one cell culture well having an opening at one end and a side wall, the at least one microfluidic channel in fluid communication with the side wall of the at least one cell culture well, and having a maximum channel width substantially less than a maximum width of the at least one cell culture well.

2. The apparatus of claim 1, wherein the one or more interconnecting layers comprise two or more interconnecting layers, wherein the at least one microfluidic channel is defined by the two or more interconnecting layers,

3. The apparatus of claim 1, wherein the maximum width of the at least one cell culture well is at least ten times the maximum channel width.

4. The apparatus of claim 1, wherein the maximum width of the at least one cell culture well is at least sixty times the maximum channel width.

5. The apparatus as in claim 1, further comprising an at least one controllable valve in fluid communication with the at least one microfluidic channel, the at least one controllable valve configured to selectably restrict fluid transport through the at least one microfluidic channel.

6. (canceled)

7. The apparatus of claim 5, further comprising at least one pump in fluid communication with the at least one microfluidic channel, the at least one pump configured to transport fluid through the at least one microfluidic channel.

8. (canceled)

9. The apparatus as in claim 1, wherein the one or more interconnecting layers comprises polydimethylsiloxane (PDMS).

10. The apparatus as in claim 1, further comprising a removable top layer adapted to cover each of the at least one cell culture well.

11. The apparatus as in claim 1, wherein the at least one cell culture well contains at least one of a pluripotent cell.

12. The apparatus as in claim 1, further comprising an externally accessible port in fluid communication with at least one of the at least one microfluidic channels.

13. The apparatus of claim 1, further comprising:
at least one sensor in proximity to the at least one cell culture well and configured to observe a physical attribute of a set of test cells disposed within the at least one cell culture well;
a controller in communication with the at least one sensor; and
at least one fluid flow regulator in communication with the controller,
wherein the controller controls the at least one fluid flow regulators thereby regulating transportation of fluid through the at least one microfluidic channel responsive to the physical attribute observed by the at least one sensor.

14. The apparatus of claim 13, further comprising a sensor selected from the group consisting of: an image sensor; a flow rate sensor; an ionic composition sensor; a temperature sensor; a pressure sensor; a light sensor, and a spectroscopic sensor.

15. A method for culturing test cells, comprising:
transferring at least one set of test cells through an opening disposed at one end of at least one cell culture well; and
transporting a fluid through at least one microfluidic channel in fluid communication with a side wall of the at least one cell culture well, each of the at least one microfluidic channels having a maximum channel width substantially less than a maximum width of the at least one cell culture well, wherein the transported fluid promotes culturing of the at least one set of test cells disposed within the at least one cell culture well.

16. The method of claim 15 wherein the act of transporting fluid through the at least one microfluidic channel comprises pumping fluid through the at least one microfluidic channel.

17-18. (canceled)

19. The method of claim 15, further comprising regulating by at least one valve the transportation of a fluid through the at least one microfluidic channel.

20. The method of claim 15, further comprising measuring at least one parameter selected from the group consisting of: a microfluidic channel fluid velocity; a microfluidic channel fluid ionic composition; a cell culture well temperature; a cell culture well pressure; optical transmissivity, optical reflectivity, and a spectroscopic data of the cell culture well.

21. The method of claim 15, further comprising reversibly sealing a removable top layer adapted to cover the opening disposed at one end of each of the at least one cell culture wells.

22. The method of claim 15, wherein at least one of the at least one set of test cells comprises at least one of a pluripotent cell.

23. The method of claim 15, further comprising:
sensing a physical attribute of the at least one set of test cells disposed within the at least one cell culture well; and
regulating transportation of fluid through the at least one microfluidic channel responsive to the sensed physical attribute.

24. An apparatus for culturing cells, comprising:
means for storing at least one set of test cells, the at least one set of test sells being transferable through an opening disposed at one end; and
means for transporting a fluid through at least one microfluidic channel in fluid communication with a side wall of the at storing means, each of the at least one microfluidic channels having a maximum channel width substantially less than a maximum width of the storing means, wherein the transported fluid promotes culturing of the at least one set of test cells disposed within the storing means.
